(19) United States
(12) Patent Application Publication
(10) Pub. No.: US 2023/0191088 A1
(43) Pub. Date: Jun. 22, 2023

(54) Device and Method for Distal Loading of Guidewires

(71) Applicant: Bard Access Systems, Inc., Salt Lake City, UT (US)

(72) Inventors: Daniel B. Blanchard, Bountiful, UT (US); Eric W. Lindekugel, Salt Lake City, UT (US)

(21) Appl. No.: 18/082,277

(22) Filed: Dec. 15, 2022

Related U.S. Application Data
(60) Provisional application No. 63/290,504, filed on Dec. 16, 2021.

(51) Int. Cl. A61M 25/09; A61M 25/00

(52) U.S. Cl. CPC A61M 25/09041; A61M 25/0026; A61M 25/0074

(57) ABSTRACT

Embodiments disclosed herein are directed to apparatus and methods for distal loading of guidewires into a lumen of a catheter, dilator, or similar elongate medical device. Distal loading of guidewires can be challenging due to the small diameter of the guidewire and the diameter of the lumen into which they are placed. Further, misalignment of the guidewire can lead to damage of delicate and complex distal tip structures. Embodiments can also include barrier and clip systems to facilitate aseptic, no-touch techniques for placing catheter and guidewire assemblies subcutaneously. A user can manipulate the catheter using the barrier, clip, and/or guidewire loading device to advance the catheter and disengage the catheter once placed.

### PRIORITY

This application claims the benefit of priority to U.S. Provisional Application No. 63/290,504, filed Dec. 16, 2021, which is incorporated by reference in its entirety into this application.

### BACKGROUND

Distal loading of guidewires into a lumen of a catheter, dilator, or similar elongate medical device can be preferable, especially where proximal loading is obstructed by the presence of access ports, hubs, valves, or similar complex proximal structures. However, the distal loading of guidewires into the lumen of an elongate medical device can be challenging due to the small diameter of the guidewire and the small diameter of the lumen into which they are placed. Misalignment of the guidewire can lead to damage of delicate and complex tip or lumen structures, and/or damage to coatings on one or both of the guidewire or medical device. In addition, handling portions of these devices, which are to enter the body during the guidewire loading process, can increase the risk of infection. Even where proximal loading of guidewires is possible, this may not be preferable since the guidewire must be advanced through an entire length of the elongate medical device. This leads to degradation of any coatings disposed on either of the guidewire or the medical device. Further, misalignment can still result in damage to one or both of the guidewire or medical device.

### SUMMARY

Briefly summarized, embodiments disclosed herein are directed to apparatus and methods for distal loading of guidewires into a lumen of a catheter, dilator, or similar elongate medical device. Embodiments facilitate alignment of the guidewire with a lumen of the elongate medical device, and provide a protective barrier between the guidewire and any structure or coating disposed thereon. Further, embodiments also include a flexible, thin-film barrier configured to mitigate direct contact with the catheter, and/or provide a sterile environment within which to contain the catheter, or portions thereof that are to be placed subcutaneously. The film barrier mitigates the introduction of pathogens, reducing the risk of infection. Embodiments further include clips configured to facilitate gripping and advancement of the catheter into the patient using aseptic, no-touch techniques.

Disclosed herein is a catheter placement system including, a catheter defining a catheter lumen, and a guidewire loading device having, a body defining a device lumen extending longitudinally, a funnel disposed at a distal end of the body and communicating with the device lumen, a slot extending longitudinally between the distal end and a proximal end of the body and communicating with the device lumen, and a protrusion extending radially inwards from an inner surface of the device lumen and configured to engage a skive disposed in an outer surface of the catheter.

In some embodiments the device lumen includes a funnel section disposed distally, a catheter section disposed proximally, and an alignment section disposed therebetween, the protrusion extending from an inner surface of the catheter section.

In some embodiments the funnel section defines a tapered inner profile extending from a first diameter at a distal end of the funnel section, to a second diameter at a proximal end of the funnel section, the second diameter being less than the first diameter.

In some embodiments the alignment section defines a tapered inner profile extending from the second diameter at a distal end of the alignment section, to a third diameter at a proximal end of the alignment section, the third diameter being equal to an inner diameter of the catheter lumen.

In some embodiments the catheter section defines a fourth diameter larger than the third diameter, a wall of the catheter section extending parallel to a longitudinal axis.

In some embodiments the fourth diameter is equal to or larger than an outer diameter of the catheter, the catheter section configured to slidably engage an outer surface of the catheter.

In some embodiments the fourth diameter is equal to or less than an outer diameter of the catheter, the catheter section configured to engage the catheter in an interference fit.

In some embodiments the catheter section further includes a tapered distal section, and inner profile of the tapered distal section mirrors an outer profile of a distal tip structure of the catheter.

In some embodiments the device lumen further includes an abutment extending radially inwards from a wall of the device lumen and configured to engage a distal tip of the catheter to inhibit further distal movement of the catheter through the device lumen.

In some embodiments the skive includes an aperture communicating with a second catheter lumen.

In some embodiments the body includes a first body portion hingedly coupled to a second body portion and pivotable through a plane extending perpendicular to a longitudinal axis.

In some embodiments the first body portion and the second body portion are transitionable between a closed position and an open position, a smallest diameter of the device lumen being larger than an outer diameter of the catheter when the device is in the open position.

In some embodiments the protrusion is configured to disengage the skive in the open position to allow the catheter to slide longitudinally relative to the body.

In some embodiments the guidewire loading device further includes a living hinge configured to bias the first body portion and the second body portion towards a closed position.

In some embodiments the catheter placement system further includes a guidewire defining an outer diameter equal to or less than the third diameter.

In some embodiments the catheter placement system further includes a barrier coupled to the guidewire loading device and extending proximally to encircle a portion of the catheter.

In some embodiments the barrier includes a tear line extending longitudinally.

In some embodiments the proximal end of the barrier is coupled to a clip configured to engage the catheter.

In some embodiments the proximal clip includes a first clip body portion hingedly coupled to a second body portion and defining a lumen, a diameter of the proximal clip lumen is larger than an outer diameter of a distal end of the barrier.

Also disclosed is a method of loading a guidewire into a lumen of a catheter including, placing a distal tip structure of the catheter within a lumen of a guidewire loading device, advancing a proximal end of a guidewire along a longitudinal axis into a funnel of the guidewire loading device, the funnel disposed at a distal end of the lumen of the guidewire loading device, slidably engaging the guidewire with a lumen of the catheter, transitioning the guidewire loading device from a closed position to an open position, and disengaging the guidewire loading device from the catheter by sliding the catheter perpendicular to the longitudinal axis, through a slot.

In some embodiments the lumen of the guidewire loading device includes a funnel section disposed distally, a catheter section disposed proximally, and an alignment section disposed therebetween.

In some embodiments the method further includes engaging the catheter with the catheter section of the lumen in one of an interference fit, press-fit or snap-fit engagement.

In some embodiments the method further includes engaging a protrusion with a skive disposed in an outer surface of the catheter when the device is in the closed position, and disengaging the protrusion from the skive when the device is in the open position, the protrusion extending radially inwards from an inner surface of the catheter section.

In some embodiments the funnel section extends from a first diameter at a distal end to a second diameter at a proximal end.

In some embodiments the alignment section extends from the second diameter at the distal end to a third diameter at the proximal end, the third diameter being equal to one or both of an inner lumen diameter of the catheter and an outer diameter of the guidewire.

In some embodiments the method further includes impinging a distal tip of the catheter against an abutment disposed within the lumen to prevent further distal movement of the catheter through the lumen of the guidewire loading device.

In some embodiments the step of transitioning further includes rotating a first arm relative to a second arm through a plane extending perpendicular to the longitudinal axis to rotate a first body portion away from a second body portion.

In some embodiments the first body portion includes a first edge and the second body portion includes a second edge, the first edge and the second edge define the slot.

In some embodiments the slot in the closed position defines a first width and the slot in the open position defines a second width, the first width being less than an outer diameter of the catheter, and the second width being larger than the outer diameter of the catheter.

Also disclosed is a method of placing a catheter including, providing a catheter placement assembly including a guidewire loading device engaged with a distal end of the catheter, a barrier, and a clip, loading a guidewire into a lumen of the catheter by advancing the guidewire longitudinally proximally into a funnel of the guidewire loading device, transitioning the guidewire loading device from a closed position to an open position, and advancing the clip distally, the clip releasably coupled to a portion of the catheter.

In some embodiments the barrier is formed of a flexible material and encircles the longitudinal axis of the guidewire loading device, a distal end of the barrier coupled with the guidewire loading device.

In some embodiments the method further includes a connector ring coupled with a distal end of the barrier and releasably engaged with a proximal end of the guidewire loading device in one of an interference fit, press-fit, snap-fit engagement, threaded engagement, bayonet engagement, or luer lock.

In some embodiments a proximal end of the barrier is coupled to the clip and is configured to maintain a sterile environment therein.

In some embodiments the step of transitioning further includes separating a cord from the barrier to rupture the barrier longitudinally along tear line.

In some embodiments the step of advancing further includes advancing a portion of the catheter through the lumen of the guidewire loading device.

In some embodiments the method further includes transitioning the guidewire loading device from an open position to a closed position to grip a first portion of the catheter, transitioning the clip from a closed position to an open position and sliding the clip proximally to grip a second portion of the catheter, to advance the catheter in iterative stages.

In some embodiments the step of advancing further includes disengaging the guidewire loading device from the catheter before advancing the clip distally.

Also disclosed is a catheter placement system including, a catheter defining a catheter lumen and extending longitudinally, a guidewire loading device including a body defining a device lumen and an arm extending from the body configured to transition the body between a closed position and an open position, and a barrier coupled to the guidewire loading device.

In some embodiments the arm is configured to be rotatable relative to the body through a plane extending parallel to the longitudinal axis.

In some embodiments the device lumen includes a funnel section, an alignment section, and a catheter section, a diameter of the alignment section is equal to one or both of an outer diameter of a guidewire and an inner diameter of the catheter lumen, a diameter of the catheter section equal to an outer diameter of the catheter.

In some embodiments the arm is configured to split the alignment section of the body along a longitudinal axis to transition the diameter of the alignment section to an outer diameter of the catheter when the device is in the open position.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a guidewire loading system including a barrier, a guidewire, and a catheter, in accordance with embodiments disclosed herein.

FIG. 2A shows a proximal end perspective view of a guidewire loading system, in accordance with embodiments disclosed herein.

FIG. 2B shows a distal end perspective view of a guidewire loading system, in accordance with embodiments disclosed herein.

FIG. 2C shows a lateral cross-section view of a guidewire loading system in a closed position, in accordance with embodiments disclosed herein.

FIG. 2D shows a lateral cross-section view of a guidewire loading system in an open position, in accordance with embodiments disclosed herein.

FIG. 3 shows a longitudinal cross-section view of a guidewire loading system, in accordance with embodiments disclosed herein.

FIG. 4A shows a perspective view of a barrier including a proximal clip for use with a guidewire loading system, in accordance with embodiments disclosed herein.

FIG. 4B shows a guidewire loading system including a barrier, a proximal clip, a guidewire, and a catheter, in accordance with embodiments disclosed herein.

FIG. 5A shows a longitudinal cross-section view of a guidewire loading system engaged with a catheter in a closed position, in accordance with embodiments disclosed herein.

FIG. 5B shows a longitudinal cross-section view of a guidewire loading system in an open position, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

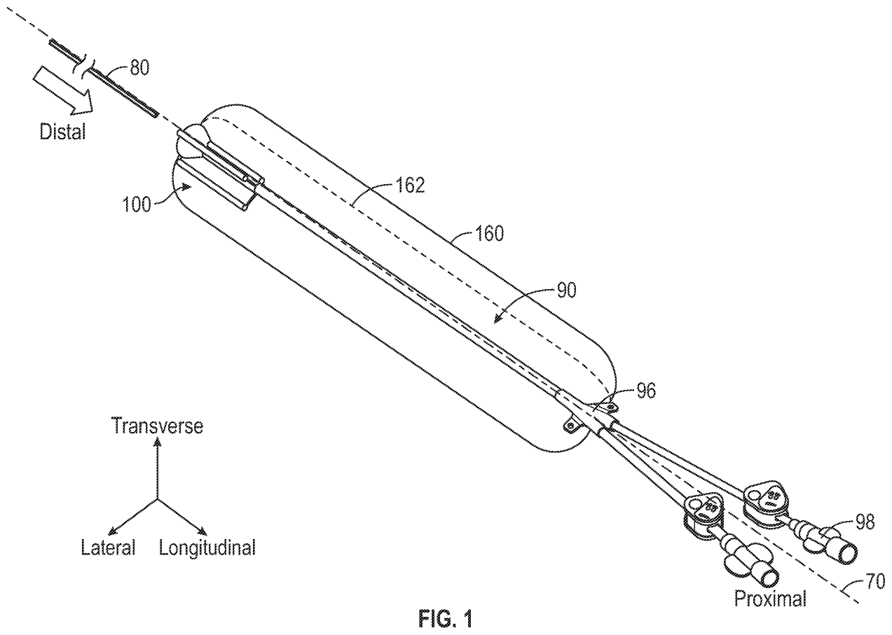

To assist in the description of embodiments described herein, as shown in FIG. 1, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

FIG. 1 shows a guidewire loading device (“device”) 100 in an exemplary environment of use. The guidewire loading device 100 can be configured to provide aseptic, no-touch techniques for distal loading of a guidewire 80 within a lumen 92 of a catheter 90. Distal loading of a guidewires into an elongate medical devices can be challenging, especially when aligning the guidewire 80 and the lumen 92. Misalignment of the guidewire 80 can lead to damage of the distal tip structure 94 of the catheter 90. Optionally, the device 100 can also prevent needle-stick injuries from a distal tip of the elongate medical device. As used herein, the catheter 90 is not intended to be limiting and the catheter 90 can include various catheters, dilators, cannulas, needles, or similar elongate medical devices configured to receive a guidewire 80 therein. In an embodiment, the device 100 can further include a barrier 160 coupled thereto and extending over a portion of the catheter 90. In an embodiment, the barrier 160 can mitigate direct contact with the catheter 90 (or similar elongate medical device). In an embodiment, the barrier 160 can maintain a sterile environment within which at least a portion of the catheter 90 is disposed.

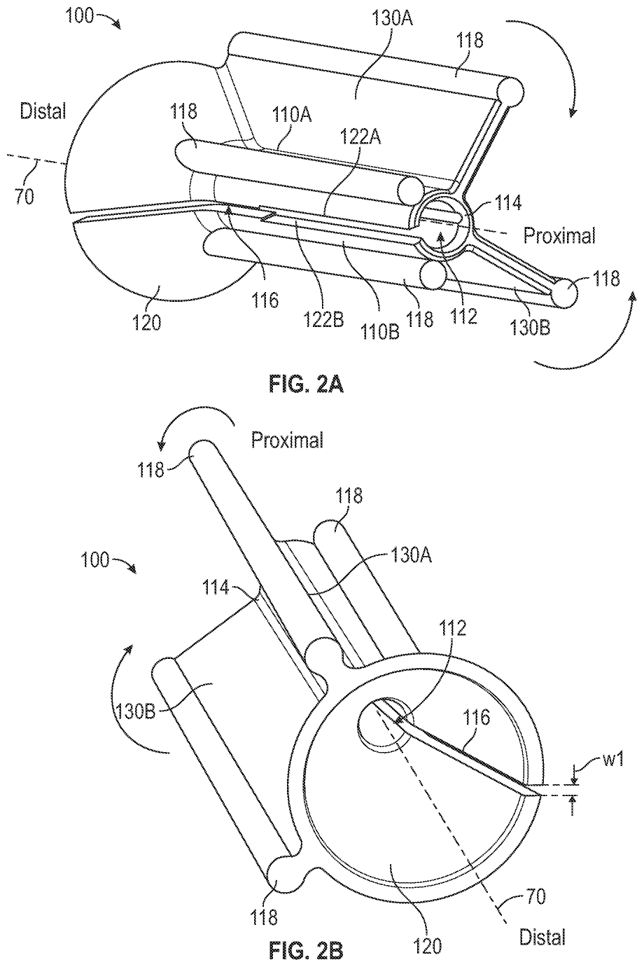

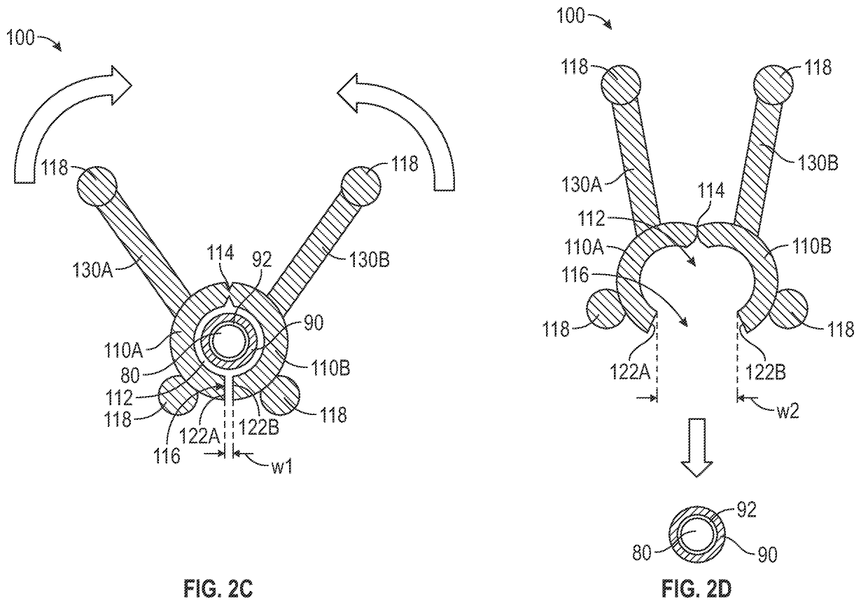

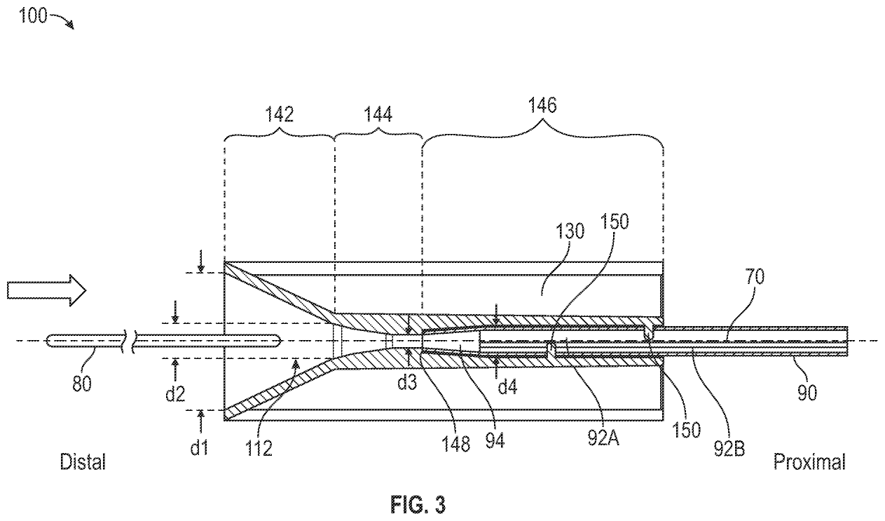

FIGS. 2A-3 show further details of the guidewire loading device 100. In an embodiment, the device 100 includes a body 110 comprising a first body portion 110A hingedly coupled to a second body portion 110B with a hinge 114. The hinge 114 can be a mechanical hinge, a living hinge, or similar mechanism configured to allow the first body portion 110A to pivot relative to the second body portion 110B through a plane extending at an angle relative to the longitudinal axis. In an embodiment, the body 110 is formed of a plastic, polymer, thermoplastic, metal, alloy, composite, or similar substantially rigid or resilient material.

In an embodiment, the first body portion 110A and the second body portion 110B co-operate to define a lumen 112 extending from a proximal end of the device 100 to a distal end of the device 100 along a central longitudinal axis 70. The device 100 further includes a slot 116 extending longitudinally between the proximal end and the distal end of the device 100 and communicating with the lumen 112. In an embodiment, the slot 116 is disposed opposite the hinge 114 across the central longitudinal axis 70. In an embodiment, the device 100 further includes a funnel 120 disposed at a distal end of the device 100. The funnel 120 defines a tapered inner profile, an apex of which is aligned with the central longitudinal axis 70 and communicates with the lumen 112.

In an embodiment, the body 110 includes one or more arms 130 extending therefrom, substantially perpendicular to the central longitudinal axis 70. FIG. 2C shows a lateral cross-section view of the body 110 in a closed position, FIG. 2D shows a lateral cross-section view of the body 110 in an open position. The first body portion 110A includes a first arm 130A, and the second body portion 110B includes a second arm 130B. The arms 130 are configured to be grasped by a user and provide mechanical advantage to transition the device 100 between the closed position (FIG. 2C) and the open position (FIG. 2D). In an embodiment, the first body portion 110A and the second body portion 110B pivot relative to each other about the hinge 114 and transition between the closed position (FIG. 2C) and the open position (FIG. 2D). In an embodiment, the device 100 can be biased towards the closed position.

In an embodiment, a first edge 122A of the first body portion 110A and a second edge 122B of the second body portion 110B define the slot 116. In an embodiment, with the device 100 in the closed position, the first edge 122A and the second edge 122B are in a spaced apart relationship such that the slot 116 defines a first width (w1) extending perpendicular to the longitudinal axis. In an embodiment, with the device 100 in the closed position, the first edge 122A contacts the second edge 122B to create a slit.

In an embodiment, with the device 100 in the open position, the first edge 122A and the second edge 122B are in a spaced apart relationship such that the slot 116 defines a second width (w2) extending perpendicular to a longitudinal axis. The second width (w2) being greater than the first width (w1). In an embodiment, the first width (w1) is equal to or less than an outer diameter of the catheter 90, to retain the catheter 90 within the device lumen 112. In an embodiment, the second width (w2) is equal to or greater than an outer diameter of the catheter 90, to allow ingress/egress of the catheter 90 to/from the device lumen 112. In an embodiment, the device 100 can further include one or more support structures 118 configured to provide increased rigidity to the device 100 during use.

FIG. 3 shows a longitudinal cross-section view of the device 100. In an embodiment, the lumen 112 includes a funnel section 142, an alignment section 144, and a catheter section 146. In an embodiment, the funnel section 142 of the lumen 112 defines a tapered inner profile extending from a first diameter (d1) at a distal end of the funnel section 142, to a second diameter (d2) at a proximal end of the funnel section 142. The second diameter (d2) being less than the first diameter (d1). The funnel section 142 defines a continuous or discontinuous change in diameter between the first diameter (d1) and the second diameter (d2).

In an embodiment, the alignment section 144 of the lumen 112 defines a tapered inner profile extending from the second diameter (d2) at a distal end of the alignment section 144 to a third diameter (d3) at a proximal end of the alignment section 144. The third diameter (d3) being less than the second diameter (d2). In an embodiment, the third diameter (d3) is equal to an inner diameter of the catheter lumen 92. The alignment section 144 defines a continuous or discontinuous change in diameter between the second diameter (d2) and the third diameter (d3). In an embodiment, a wall of the funnel section 142 defines a steeper angle relative to the longitudinal axis, relative to an angle of a wall of the alignment section 144. In an embodiment, an angle of a wall of the alignment section 144 extends substantially parallel to the longitudinal axis.

In an embodiment, the catheter section 146 of the lumen 112 defines a fourth diameter (d4) which is greater than the third diameter (d3) and equal to or less than the first diameter (d1). In an embodiment, a wall of the catheter section 146 extends substantially parallel to a longitudinal axis. In an embodiment, a distal portion of the catheter section 146 defines a tapered inner profile configured to mirror an outer profile of a distal tip structure 94 of the catheter 90. The tapered inner profile of the catheter section 146 extends distally from the fourth diameter (d4) to the third diameter (d3).

In an embodiment, the fourth diameter (d4) is equal to, or slightly larger than, an outer diameter of the catheter 90. In an embodiment, the fourth diameter (d4) is equal to, or slightly smaller than, an outer diameter of the catheter 90, to engage the catheter 90 in a friction fit engagement. In an embodiment, an inner surface of the catheter section 146 includes one or more protrusions 150 extending radially inwards. The protrusions 150 are configured to engage a skive or an aperture disposed in a side wall of the catheter 90 to secure a portion of the catheter 90 within the catheter section 146 in a snap-fit engagement. In an embodiment, the skive is a recess or detent disposed in an outer surface of the catheter 90. In an embodiment, the skive is an aperture communicating with a lumen 92 of the catheter, for example a first catheter lumen 92A or a second catheter lumen 92B. As such, the protrusion 150 mitigates disengagement of the device 100 from the catheter 90 as the guidewire 80 is urged proximally into the catheter lumen 92. In an embodiment a proximal end of the device lumen 112 includes a chamfered or beveled edge configured to facilitate aligning a distal portion of the catheter 90 therewith.

In an embodiment, a distal portion of the catheter 90 is received within the catheter section 146 of the lumen 112. The device 100 engages the catheter 90 in an interference fit, press-fit, or friction fit engagement to retain the catheter 90 within the catheter section 146 of the lumen 112. In an embodiment, the protrusions 150 engage a skive, or aperture, disposed in the catheter 90 to retain the catheter 90 within the catheter section 146 in a snap-fit engagement. In an embodiment, a distal end of the catheter section 146 includes an abutment 148 configured to engage a distal end of the catheter 90. The catheter 90 is advanced distally into the catheter section 146 until a distal tip of the catheter 90 abuts against the abutment 148, preventing further advancement thereof. Further, the catheter section 146 is configured to align a distal opening of the catheter lumen 92 with the proximal end of the alignment section 144. As noted, the third diameter (d3) is equal to the diameter of the catheter lumen 92 at the distal opening.

In an embodiment, a guidewire 80 is inserted into a distal end of the lumen 112. The outer diameter of the guidewire 112 being equal to or less than an inner diameter of the catheter lumen 92, i.e. equal to or less than the third diameter (d3). As the guidewire 80 is advanced proximally into the device 100, the funnel section 142 aligns a proximal end of the guidewire 80 with the alignment section 144. Further, the alignment section 144 aligns the proximal end of the guidewire 112 with the catheter lumen 92. As such, the device 100 facilitates loading the guidewire 80 into a distal end of the catheter 90 while protecting the distal tip structure 94 of the catheter 90.

With continued reference to FIGS. 2A-2D, with the guidewire 70 loaded within the catheter lumen 92, a user can actuate the arms 130 to transition the device 100 from the closed position to an open position to allow the device 100 to disengage from the catheter 90 and guidewire 70 assembly. A user can apply an opposing, “pinching” forces to the first arm 130A and the second arm 130B, pivoting the arms 130A, 130B towards each other and rotating the body portions 110A, 110B about the hinge 144, transitioning the device 100 to the open position.

In an embodiment, the first edge 122A of the first body portion 110A and the second edge 122B of the second body portion 110B that define the slot 116 rotates apart from each other through a plane extending perpendicular to the longitudinal axis. In the open position the width of the slot 116 increases from the first width (w1) to the second width (w2). In an embodiment, the second width (w2) is greater than an outer diameter of the catheter, i.e. equal to or greater than diameter (d4), allowing the catheter 90 and guidewire 70 assembly to pass laterally through the slot 116 and disengage the device 100.

In an embodiment the hinge 114 is malleable and configured to remain in the open position until repositioned by a user. In an embodiment, the device 100 is biased to the closed position. As such, when a user releases the arms 130, the device 100 returns to the closed position. In an embodiment, the hinge 114 is a living hinge formed from a resilient material and can bias the device 100 towards the closed position. In an embodiment one or both of the first edge 122A and the second edge 122B includes a magnetic material (e.g. permanent magnet, ferrous material, magnetizable material, etc.) configured to bias the device 100 to the closed position.

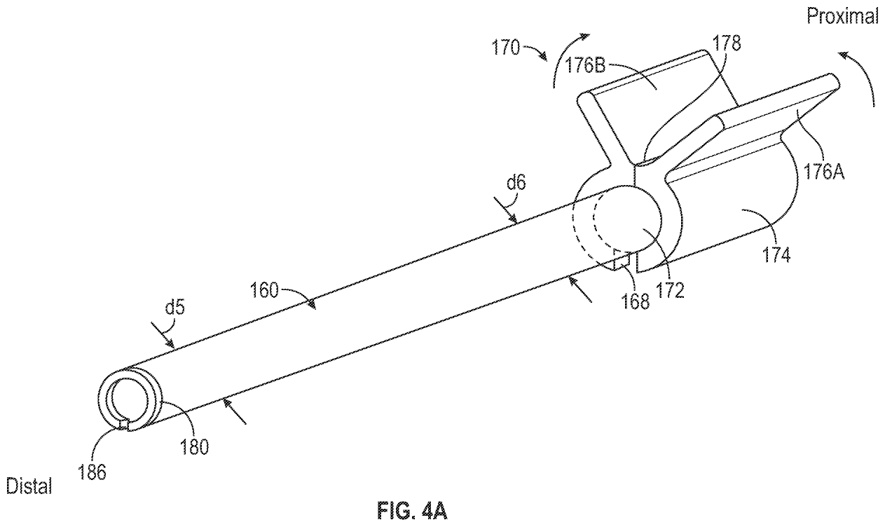

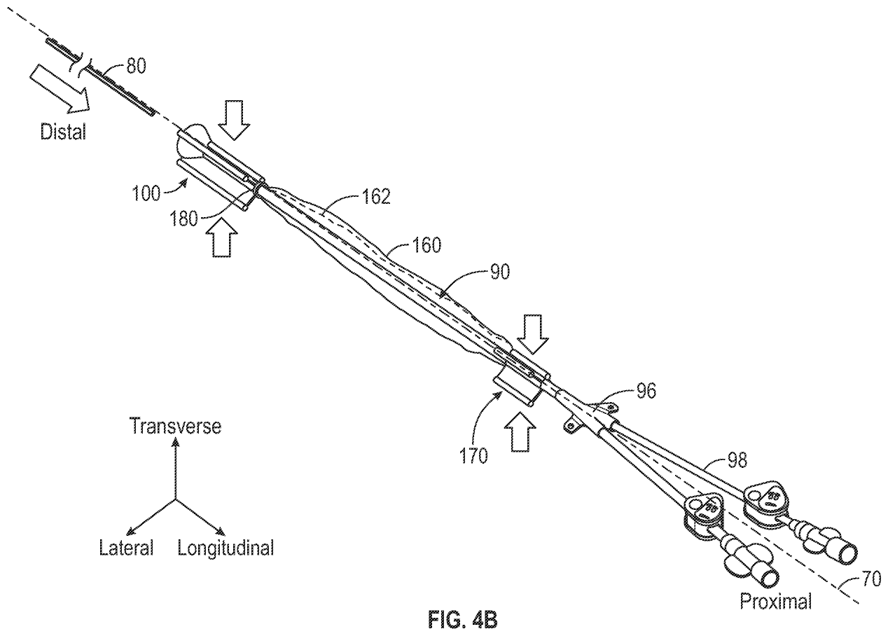

In an embodiment, as shown in FIGS. 1 and 4A-4B, the device 100 further includes a barrier 160 formed of a flexible, thin-film material and extending about the central longitudinal axis 70 of one or both of a portion of the device 100 and at least a portion of the catheter 90. As such, the barrier 160 mitigates direct-touch contact with the catheter 90, or portions of the catheter 90 that are designed to be disposed within the body of the patient. In an embodiment, the barrier 160 is formed of a flexible film, gas-impermeable, and/or polymeric material, and is configured to maintain a sterile environment therein. In an embodiment, the barrier 160 is formed as an extruded, substantially cylindrical shape and is coupled to the device 100 to encircle the central lumen 112 of the device 100 and/or the catheter 90.

In an embodiment, a distal end of the barrier 160 is coupled to a distal end of the body 110 and encloses both the device 100 and a portion of the catheter 90 therein. In an embodiment, a distal end of the barrier 160 is coupled to a proximal end or a mid-point of the body 110 of the device 100. In an embodiment, the proximal end of the barrier 160 defines an opening, allowing a portion of the catheter 90 to extend therethrough. In an embodiment, the proximal end of the barrier 160 is coupled to a portion of the catheter 90, for example a hub 96, or extension leg 98. As such, the barrier 160 encloses a portion of the catheter 90, e.g. a portion that is to be placed within a patient's body, within a sterile environment, mitigating the introduction of pathogens. In an embodiment, a proximal end of the barrier 160 extends proximally of a proximal end of the catheter 90 to enclose the entire catheter 90 within the sterile environment defined by the barrier 160.

In an embodiment, the barrier 160 includes a tear line 162 extending longitudinally. The tear line 162 includes a groove, score line, perforation, laser cut line, or similar line of weakness configured to allow the barrier 160 to separate therealong as the device 100 is transitioned to the open position. In an embodiment, the tear line 162 includes a cord extending therealong. Separating the cord from the barrier 160 causes the barrier 160 to rupture therealong, facilitating separation of the barrier 160. In an embodiment, the barrier 160 is formed as a sheet of material with a first longitudinal edge coupled to a second longitudinal edge, opposite the first longitudinal edge, to form the tear line 162. The first longitudinal edge can be coupled to the second longitudinal edge using an adhesive, bonding, welding, or the like to form the tear line 162. This can simplify the manufacturing process of the barrier 160.

In an embodiment, transitioning the device 100 to the open position causes a first portion of the barrier 160 coupled to the first body portion 110A and a second portion of the barrier 160 coupled to the second body portion 110B to separate along the tear line 162 and allow the catheter 90 and guidewire 70 assembly to pass therebetween. The device 100 including the barrier 160 can then be disengaged from the catheter 90.

In an embodiment, as shown in FIGS. 4A-4B, the barrier 160 includes a clip 170 coupled with a proximal end of the barrier 160 and configured to engage a portion of the catheter 90 in a similar manner to the guidewire loading device 100. The clip 170 includes a body 174 defining a clip lumen 172 and includes one or more arms 178. The body 174 further includes a hinge 178 extending longitudinally and configured to allow the body 174 to transition between an open position and a closed position when opposing “pinching” forces are applied to the arms 178, as described herein, (e.g. see FIGS. 2C-2D). In the open position, a slot 168 disposed opposite the hinge 178, across the clip lumen 172, can open to allow ingress/egress of the catheter 90 to/from the clip lumen 172. Ingress or egress of the catheter 90 can be along an axis extending perpendicular to the longitudinal axis.

In an embodiment, the clip 170 engages a proximal portion of the catheter 90, for example adjacent the hub 96. A portion of the catheter 90 extends through the clip lumen 172. In an embodiment, the clip 170 releasably engages the hub 96 in one of an interference fit, press-fit, snap-fit engagement, or similar suitable engagement mechanism. In an embodiment, a proximal end of the barrier 160 is coupled with a distal end of the clip lumen 172. In an embodiment, a distal end of the barrier 160 is coupled to the device 100, as described herein. In an embodiment, a distal end of the barrier 160 includes a connector ring 180 configured to releasably engage the device 100 in one of an interference fit, press-fit, or snap-fit engagement. In an embodiment, the connector ring 180 releasably engages the device 100 in a threaded engagement, bayonet engagement, luer lock, or by a similar suitable connection mechanism. In an embodiment, the connector ring 180 is splittable to allow for lateral separation of the connector ring 180 from a portion of the catheter 90, extending therethrough, when the device 100 is transitioned to the open position.

In an embodiment, a distal end of the barrier 160 defines a first outer diameter (d5) and a proximal end of the barrier 160 defines a second outer diameter (d6), larger than the first out diameter (d5). In an embodiment, the catheter 90 is advanced distally, through the connector ring 180 at a distal end of the barrier 160 by grasping the proximal clip 170 and urging the clip 170 longitudinally distally. To note, the clip 170 is coupled to the catheter 90, e.g. at a hub 96, or grips an outer surface of a shaft of the catheter 90 in an interference fit engagement. As such, the flexible barrier 160 collapses in on itself to allow the catheter 90 to advance distally. Since the first outer diameter (d5) is less than the second outer diameter (d6), the barrier 160 collapses into the clip lumen 172 containing the barrier 160 in a collapsed state within the body 174 of the clip 170. As such, the catheter 90 is advanced distally using aseptic, no touch techniques.

In an embodiment, once the guidewire 80 is loaded, a user can advance the catheter 90 using aseptic, no-touch techniques. For example, a user actuates the arms 130 to transition the device 100 from a closed position to an open position. In the open position, the first body portion 110A and the second body portion 110B rotate outwards such that the device lumen 112 at the distal end of the catheter section 146 separates to a width greater than the outer diameter of the catheter 90. As such, the catheter 90 can slide longitudinally through the lumen 112 such that the distal tip structure 94 extends distally of the abutment 148. The user can then grasp the proximal clip 170 and manipulate the catheter 90 to advance the catheter 90 distally through the device lumen 112. As the catheter 90 is urged distally, the barrier 160 collapses into the clip lumen 172 and is contained therein.

In an embodiment, the user can manipulate the device 100 and the clip 170 to advance the catheter 90 in a step-wise manner using aseptic, no-touch techniques. For example, in an embodiment, the clip 170 grips a mid-point of the catheter 90, i.e. at a point between the distal tip structure 94 and the hub 96. The user then actuates the arms 130 of the device 100 to widen the lumen 112, as described herein. The user then grasps the clip 170 and advances a portion of the catheter 90 through the device lumen 112. The user then releases the arms 130 and the device 100, which is biased towards the closed position, grips the catheter 90 within the device lumen 112. The user then actuates the arms 176 of the clip to release the catheter 90 and slide the clip proximally. The user releases the arms 176 of the clip and transitions the clip 170 from the open position to the closed position. Optionally, the clip 170 is also biased towards the closed position. The user then grips a second portion of the catheter 90 and repeats the process to advance the catheter 90 through the device lumen 112 in iterative stages.

In an embodiment, the device 100 including one of the barrier 160 and the connection ring 180 disengages the catheter 90 by urging the catheter 90 through the slot 116 while the device 100 is in the closed position. In an embodiment, the connection ring 180 includes a slot 186 that aligns with the slot 116 of the device 100 when coupled thereto. In an embodiment, the device 100 including one of the barrier 160 and the connection ring 180 disengages the catheter 90 by transitioning the device 100 to the open position and optionally splitting one or both of the connection ring 180 (i.e. at slot 186), and the barrier 160 along the tear line 162. The catheter 90 then slides laterally through the slot 116.

In an embodiment, the user disengages the device 100 from the catheter 90 before advancing the catheter 90 distally into the patient. As such, with the device 100 and distal end of the barrier 160 disengaged from the catheter 90, the user then grasps the clip 170 to advance the catheter 90 distally into the patient. The barrier 160 separates along the tear line 162 as the catheter 90 is advanced. Once the catheter 90 is placed within the patient, the user actuates the proximal clip arms 176 to pivot the clip body 174 to the open position. The clip 170 then disengages the catheter 90 by passing the catheter 90 laterally through the clip slot 168.

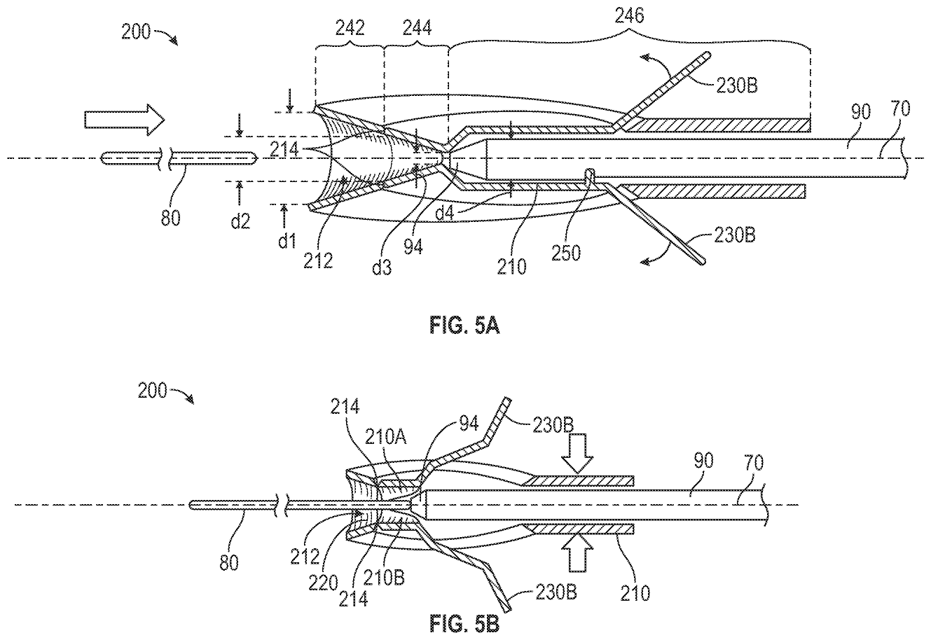

FIGS. 5A-5B show an embodiment of a guidewire loading device (“device”) 200 configured to engage a distal portion of the catheter 90 and facilitate distal loading of a guidewire 80, as described herein. In an embodiment, the device 200 includes a body 210 defining a lumen 212 having one or more of a funnel section 242, alignment section 244, and a catheter section 246. In an embodiment, the device 200 includes one or more arms 230 coupled to the device 200. The arms 230 are configured to pivot relative to the body 210 through a plane extending parallel to the central longitudinal axis 70 to transition the device 200 between a closed position (FIG. 5A) and an open position (FIG. 5B).

In an embodiment, the catheter section 246 of the lumen 212 engages a distal portion of the catheter 90 in one of an interference fit, press-fit, or snap-fit engagement, as described herein. In an embodiment, the device 200 further includes one or more protrusions 250 configured to engage a skive or aperture disposed in the catheter 90 and retain the distal tip structure 94 of the catheter 90 within the catheter section 246, as described herein. With the catheter tip structure 94 secured within the catheter section 246, the guidewire 80 can be advanced through the funnel section 242 and the alignment section 244 to align the guidewire 80 with the catheter lumen 92 and advance the guidewire 80 proximally into the catheter lumen 92.

In an embodiment, the device 200 is transitioned from the closed position (FIG. 5A) to the open position (FIG. 5B), by rotating the arms 230 outwards, through a plane extending parallel to the central longitudinal axis 70. In an embodiment, in the open position, the protrusions 250 disengage the skives, releasing the catheter 90. In an embodiment, transitioning the arms 230 to the open position cause a portion of the body 210 to split along a longitudinal axis and allow the device 200 to disengage the catheter 90 and guidewire 80 assembly. In an embodiment, in the open position the arms 230 split the alignment section 244 along a longitudinal axis into a first body portion 210A and a second body portion 210B. Each of the first body portion 210A and the second body portion 210B pivot about a hinge 214 coupling a distal end of the first body portion 210A and the second body portion 210B, respectively, to the funnel 220. As such, the proximal end of the alignment section 244 can expand radially outward from the third diameter (d3) to a diameter equal to or greater than the outer diameter of the catheter 90, i.e. diameter (d4), allowing for distal advancement of the catheter 90 through the device lumen 212.

In an embodiment, with the device 200 in the open position, a user pinches a proximal end of the body 210 adjacent a catheter section 246, and elastically deforms a portion of the body 210 to grip the catheter 90 disposed therein. The user then manipulates the catheter 90 in an aseptic, no-touch technique by gripping the catheter within the lumen 212. The user then releases the body 210 and in turn releases the catheter 90 and slides the body 210 proximally over the catheter 90. The user then repeats the process to grasp a second portion of the catheter 90 and continues to advance the catheter 90. As such, the user advances the catheter 90 in an iterative process without touching the catheter 90. In an embodiment, the device 200 further includes one or more of the barrier 160, clip 170, and connector 180, to prevent direct contact with the catheter 90 or provide a sterile environment, while facilitating aseptic, no touch catheter placement techniques, as described herein.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter placement system, comprising:
a catheter defining a catheter lumen; and
a guidewire loading device, comprising:
a body defining a device lumen extending longitudinally;
a funnel disposed at a distal end of the body and communicating with the device lumen;
a slot extending longitudinally between the distal end and a proximal end of the body and communicating with the device lumen; and
a protrusion extending radially inwards from an inner surface of the device lumen and configured to engage a skive disposed in an outer surface of the catheter.

2. The catheter placement system according to claim 1, wherein the device lumen includes a funnel section disposed distally, a catheter section disposed proximally, and an alignment section disposed therebetween, the protrusion extending from an inner surface of the catheter section.

3. The catheter placement system according to claim 2, wherein the funnel section defines a tapered inner profile extending from a first diameter at a distal end of the funnel section, to a second diameter at a proximal end of the funnel section, the second diameter being less than the first diameter.

4. The catheter placement system according to claim 3, wherein the alignment section defines a tapered inner profile extending from the second diameter at a distal end of the alignment section, to a third diameter at a proximal end of the alignment section, the third diameter being equal to an inner diameter of the catheter lumen.

5. The catheter placement system according to claim 4, wherein the catheter section defines a fourth diameter larger than the third diameter, a wall of the catheter section extending parallel to a longitudinal axis.

6. The catheter placement system according to claim 5, wherein the fourth diameter is equal to or larger than an outer diameter of the catheter, the catheter section configured to slidably engage an outer surface of the catheter.

7. The catheter placement system according to claim 5, wherein the fourth diameter is equal to or less than an outer diameter of the catheter, the catheter section configured to engage the catheter in an interference fit.

8. The catheter placement system according to claim 2, wherein the catheter section further includes a tapered distal section, and inner profile of the tapered distal section mirrors an outer profile of a distal tip structure of the catheter.

9. The catheter placement system according to claim 1, wherein the device lumen further includes an abutment extending radially inwards from a wall of the device lumen and configured to engage a distal tip of the catheter to inhibit further distal movement of the catheter through the device lumen.

10. The catheter placement system according to claim 1, wherein the skive includes an aperture communicating with a second catheter lumen.

11. The catheter placement system according to claim 1, wherein the body includes a first body portion hingedly coupled to a second body portion and pivotable through a plane extending perpendicular to a longitudinal axis.

12. The catheter placement system according to claim 11, wherein the first body portion and the second body portion are transitionable between a closed position and an open position, a smallest diameter of the device lumen being larger than an outer diameter of the catheter when the device is in the open position.

13. The catheter placement system according to claim 12, wherein the protrusion is configured to disengage the skive in the open position to allow the catheter to slide longitudinally relative to the body.

14. The catheter placement system according to claim 11, wherein the guidewire loading device further includes a living hinge configured to bias the first body portion and the second body portion towards a closed position.

15. The catheter placement system according to claim 4, further including a guidewire defining an outer diameter equal to or less than the third diameter.

16. The catheter placement system according to claim 1, further including a barrier coupled to the guidewire loading device and extending proximally to encircle a portion of the catheter.

17. The catheter placement system according to claim 16, wherein the barrier includes a tear line extending longitudinally.

18. The catheter placement system according to claim 16 wherein the proximal end of the barrier is coupled to a clip configured to engage the catheter.

19. The catheter placement system according to claim 18, wherein the proximal clip includes a first clip body portion hingedly coupled to a second body portion and defining a lumen, a diameter of the proximal clip lumen is larger than an outer diameter of a distal end of the barrier.

20-37. (canceled)

38. A catheter placement system, comprising:
a catheter defining a catheter lumen and extending longitudinally;
a guidewire loading device including a body defining a device lumen and an arm extending from the body configured to transition the body between a closed position and an open position; and
a barrier coupled to the guidewire loading device.

39. The catheter placement system according to claim 38, wherein the arm is configured to be rotatable relative to the body through a plane extending parallel to the longitudinal axis.

40. The catheter placement system according to claim 38, wherein the device lumen includes a funnel section, an alignment section, and a catheter section, a diameter of the alignment section is equal to one or both of an outer diameter of a guidewire and an inner diameter of the catheter lumen, a diameter of the catheter section equal to an outer diameter of the catheter.

41. The catheter placement system according to claim 40, wherein the arm is configured to split the alignment section of the body along a longitudinal axis to transition the diameter of the alignment section to an outer diameter of the catheter when the device is in the open position.
